(19) United States
(12) Patent Application Publication
(10) Pub. No.: US 2013/0060207 A1
(43) Pub. Date: Mar. 7, 2013

(54) METHOD AND APPARATUS FOR SINGLE-USE CREAM OR LIQUID DISPENSER AND APPLICATOR

(71) Applicant: Tamela D. Hill, Olathe, KS (US)

(72) Inventor: Tamela D. Hill, Olathe, KS (US)

(21) Appl. No.: 13/231,631

(22) Filed: Sep. 13, 2011

Related U.S. Application Data
(60) Provisional application No. 61/531,889, filed on Sep. 7, 2011.

(51) Int. Cl. A61M 35/00

(52) U.S. Cl. 604/290; 604/311

(57) ABSTRACT

A single-use disposable applicator and dispenser of creams, liquids, and spreadable agents. A liquid or cream agent of liquid or viscous is stored in a sterile container. An applicator including a soft interface is stored along with the agent. When the need to apply the agent occurs, the applicator breaks the seal to the agent, after which the applicator is used to apply the cream or liquid agent to a desired surface, and is then discarded. The invention may be used by healthcare providers and similar persons to treat a plurality of persons, and lessens the likelihood that bacteria or infection will be spread amongst a population. The invention may further be used to contain, dispense, and apply any manner of spreadable agents of low rigidity including, but not limited to, soaps, cleaning agents, and paint samples.

### CROSS-REFERENCE TO RELATED APPLICATION

This application claims priority in U.S. Provisional Patent Application No. 61/531,889, filed Sep. 7, 2011, which is incorporated herein by reference.

### BACKGROUND OF THE INVENTION

1. Field of the Invention

The present invention relates generally to a one-time use dispenser and applicator for applying a liquid or cream to a surface, such as human skin.

2. Description of the Related Art

Many individuals are afflicted with skin disorders such as eczema, psoriasis, skin allergies, and many forms of rash-inducing conditions. Additionally, many individuals suffer from allergic reactions to poison ivy and other common skin-irritating substances. There is a wide range of treatment agents for these conditions, such as steroid lotions and ointments or salves containing Vitamin E, aloe and cortisone, among many others. These agents are most frequently packaged in tubes with caps, like a tube of toothpaste. The tube containers themselves may be constructed of a variety of materials from plastic to metals and with varying rigidity. There are multiple problems with such packaging. First, caps frequently become dirty and clogged with unused or excess agent. Not only is this wasteful, but it also makes the dispenser unsanitary to use. Second, many such dispenser tubes are large and/or cumbersome when attempting to use them one-handed, in tight quarters, or quickly and on the go. In many cases, one may find him or herself wishing for an extra hand.

There are additional problems with the size of traditional packaging. First the tubes are often large and add to the bulk that individuals must carry with them on a daily basis. With specific regard for parents of diaper-aged children suffering from diaper rash, these tubes are added weight and volume to already-full diaper bags. This problem is not limited to diaper-rash creams and overburdened parents, but modern life has led to more and more individuals carrying many medications, ointments, lotions, medicaments, and any number of other liquid items with them on a daily basis. Therefore, many agents in traditional packaging create extra weight and bulk in already-crowded bags. The current state air travel also limits the amount of luggage passengers carry. Fees for checking luggage result in more carry-on baggage. Lotions, liquids and creams in carry-on baggage that one person may carry are required to be limited in volume and amount. In addition to treatments and agents for diaper rash, other ointments, and make-up, which are used topically, items such as shoe polish, paint samples, and any number of other liquids and/or creams may come in cumbersome, bulky, or wasteful packaging. Many such items, an exhaustive list of which would be too lengthy to include here, would benefit from a small, compact, single-use packaging alternative.

Aside from the issues with existing packaging, there is a concern when touching irritated or affected areas of human skin. For example, when spreading ointment onto a portion of skin affected by poison ivy, the worry is that the person applying the ointment may spread the rash to other portions of the body or to other persons. Similarly, when applying ointment to a baby suffering from diaper rash, a number of cleanliness issues arise, and the spread of rotavirus is a constant concern. Using a hand to spread cream to an affected portion of skin increases these risks and others.

What is needed is a method of applying creams or liquids to an affected area in a simple and comfortable manner, while being as sterile as possible. The present invention presents such a solution, whereby a single-use applicator may be used to treat an affected portion of human skin gently, and then the single-use applicator may be discarded.

Heretofore there has not been available a single-use applicator with the features and elements of the present invention.

### BRIEF SUMMARY OF THE INVENTION

The present invention relates to a single-use disposable applicator and dispenser of creams, liquids, and spreadable agents for the purpose of applying the agent to a desired surface. A liquid, cream, or spreadable agent of liquid or viscous is stored in a sterile container. An applicator including a soft interface is stored along with the agent. When the need to apply the agent occurs, the applicator breaks the seal to the agent, after which the applicator is used to apply the cream or liquid agent to desired body, and is then discarded.

An exemplary embodiment of the present invention addresses the multiple needs for providing a one-time use applicator and dispenser for creams, lotions, ointments, other remedies and medicaments, and any other liquid. The exemplary embodiment of the invention is sanitary, small, light-weight, and easy to pack and carry. The exemplary embodiment may be used with one hand. It will be understood by one of ordinary skill in the art that the above description is for example only, and not meant as a limitation, and any dispenser and applicator of the types described above may be used and are included in this disclosure.

One embodiment of the present invention may be used to treat general skin irritations, such as diaper rash or poison ivy rash. A healthcare provider may use an embodiment of the present invention to apply a treatment cream or ointment to the affected portion of human skin without coming into direct skin-on-skin contact with the affected area. This embodiment prevents the spread of infection or bacteria from one patient to the next because the applicator is discarded once treatment is applied. A separate sterile applicator is used for each afflicted person.

### BRIEF DESCRIPTION OF THE DRAWINGS

The drawings constitute a part of this specification and include exemplary embodiments of the present invention illustrating various objects and features thereof.

FIG. 1 is an isometric view of an embodiment of the present invention showing the embodiment within a typical environment.

FIG. 2 is a side elevation view showing an embodiment of the present invention as it would be packaged for consumption.

FIG. 3 is a side elevation view showing an embodiment of the present invention in its original position.

FIG. 4 is a side elevation view showing an embodiment of the present invention in its final position.

FIG. 5 is a side elevation as indicated by the arrow of FIG. 1, showing the forward face of an embodiment of the present invention.

FIG. 6 is a section view showing the internal workings of the present invention in its original position.

FIG. 7 is a section view showing the internal workings of the present invention after a first seal is perforated.

FIG. 8 is a section view showing the internal workings of the present invention after a second seal is perforated.

FIG. 9 is a section view showing the internal workings of the present invention in its final position.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

An embodiment of the present invention is generally designed to provide a sanitary and germ-free apparatus and method for applying liquid, cream-based, or otherwise spreadable agents onto a body, such as human skin. Some examples of such applications include, but are not limited to: burn ointment for treating mild to severe burns; hormone replacement creams; creams and ointments for treating fungal or bacterial infections of the skin, such as ring worm; and creams and ointments for treating rashes which may easily be spread, such as poison ivy.

An embodiment of the present invention would be extremely useful in areas where a number of persons may need to be treated by a very few individuals for a number of skin-related issues. This would include hospitals, daycares, nursing homes, and veterinary clinics. The present invention would also be valuable for use in handling weather emergencies or other catastrophic events, such as plane or ship wrecks, or for use in emergency medical kits. In such environments a single individual may be required to treat a number of individuals in short succession. Using a disposable applicator, such as the present invention, would decrease the likelihood that bacteria or disease would be spread from one individual to the next.

The present invention would not be limited to only those liquids, creams, and spreadable agents used to treat human skin. The present invention may be used to apply and spread any similar agent, such as glue, harsh cleaning chemicals such as tile and grout cleaner, deodorants, jewelry cleaner, and even make-up samples for use at a department store make-up counter. The applications possible with the present invention are near limitless.

### II. Example

### Treatment of Diaper Rash

One primary example of how an embodiment of the present invention may be used is in the treatment of common diaper rash in infants. The skin irritation caused by diaper rash may predispose an infant to irritation and infection. The two most common types of infection are those associated with yeast and bacteria. The most common yeast infection is caused by Candida albicans. Meanwhile, the most common bacterial infection is caused by Staphylococcus aureus.

Treatment for diaper rash involves removing the source of irritation, reducing the immediate skin reaction, relieving the discomfort, and preventing secondary infection and other complications. Frequent diaper changes and keeping the area as dry and clean as possible normally prove helpful. Keeping the area clean and dry, however, has not proven to be fully effective in protecting sensitive and/or already irritated skin from the chemical irritation associated with the by-products of infant urine and feces.

The primary issue in the treatment of diaper rash is that the caretaker who is applying standard creams and treatments is exposed to infant urine and feces, as well as any skin-borne bacteria that may be present. In a daycare facility, this can lead to a strain of infection that can be spread amongst a number of children if proper sanitary precautions are not taken

An embodiment of the present invention allows a caretaker to use an individually sealed dispenser and applicator to treat one child's diaper rash. Using the applicator, the caretaker's hands and skin do not come into direct contact with the dangerous bacteria that may be present on the child's skin. Once the applicator has been used, it is disposed of, and a second dispenser and applicator may be used on a second child. This prevents the spread of disease and infection amongst the population of children at a daycare facility.

A preferred embodiment of the present invention is of such dimensions to allow for several dispenser and applicator devices to be carried on one's person or stored in a standard hand bag. Each apparatus is individually packaged, and may be purchased or stored in a desired number. The apparatus is compact to allow a healthcare provider to carry several at one time. This eliminates the need to carry large bottles of cream or ointment.

### III. Preferred Embodiment or Aspect Single-Use Applicator 2

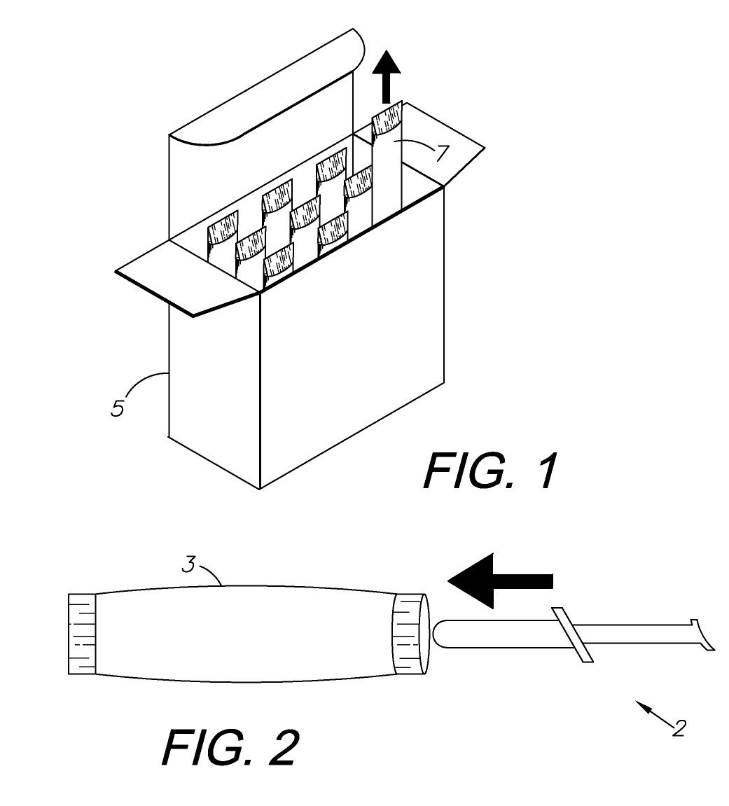

Referring to the drawings in more detail, FIG. 1 shows a typical storage container 5, such as a box containing flaps, which may contain and store a number of pre-wrapped single-use applicators 7. The applicators may be purchased in bulk for mass consumption, such as for use in a nursery for treating diaper rash, or in a medical facility for treating burns, poison ivy, or other skin irritations.

FIG. 2 shows a single use applicator 2 as it would be housed within a typical disposable wrapper 3. The wrapper may be made from paper, plastic, or any similar material which allows for easy-opening while maintaining the sterility of the applicator 2.

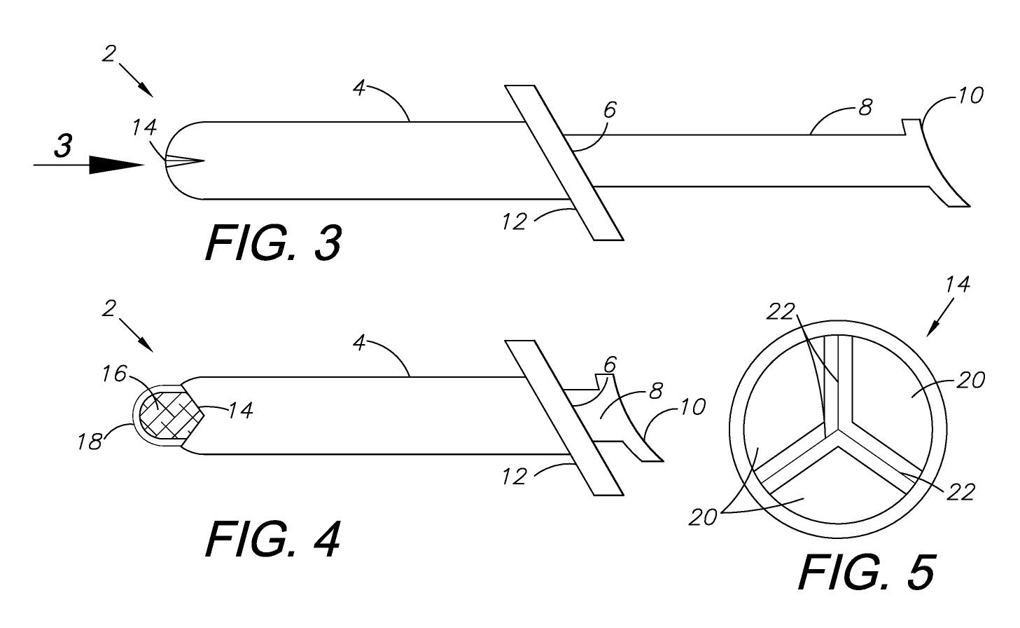

FIGS. 3 and 4 demonstrate the typical functionality and utility of an embodiment of the present invention. The single use applicator typically is comprised of a housing tube 4 which houses the applicator plunger 8 and tip 16 and is capable of storing a liquid or viscous agent 18. The applicator tip 16 is inserted into the housing tube 4 through a housing tube pathway 6. A depressor 10 at the distal end of the applicator plunger 8 allows for the applicator plunger 8 to easily travel down the pathway 6. The depressor 10 may be ergonomically formed to conform to the thumb of a human hand.

The housing tube 4 may include a grip 12 at the distal end, which is designed to receive fingers of a hand while the thumb depresses the depressor 10, guiding the applicator plunger 8 down the pathway 6. The housing tube 4, grip 12, applicator plunger 8, and depressor 10 may be created from plastic or any light-weight, sterile material.

FIG. 4 demonstrates the applicator tip 16 emerging from a front opening enclosure 14 located within the housing tube 4. The applicator tip 16 emerges from the housing tube 4 saturated with a liquid or cream agent 18 which was previously stored within the housing tube 4. At this point, the applicator 2 is in its “final position,” wherein it may apply a liquid or cream liquid or viscous agent 18 to a body using the applicator tip 16.

FIG. 5 demonstrates one way in which a front opening enclosure 14 may be created, although such an enclosure is unnecessary as long as the seals 24, 26 are in tact. A number of opening flaps 20 form a rounded tip. These flaps seal tight and may maintain a sterile seal within the housing tube 4. When pressed against from within, the opening flaps 20 open along a number of opening flap incisions 22. The opening flaps peel apart, allowing the applicator tip 16 to exit the housing tube 4, and the tip will then be free to apply the liquid or cream agent 18 to a body, such as irritated human skin.

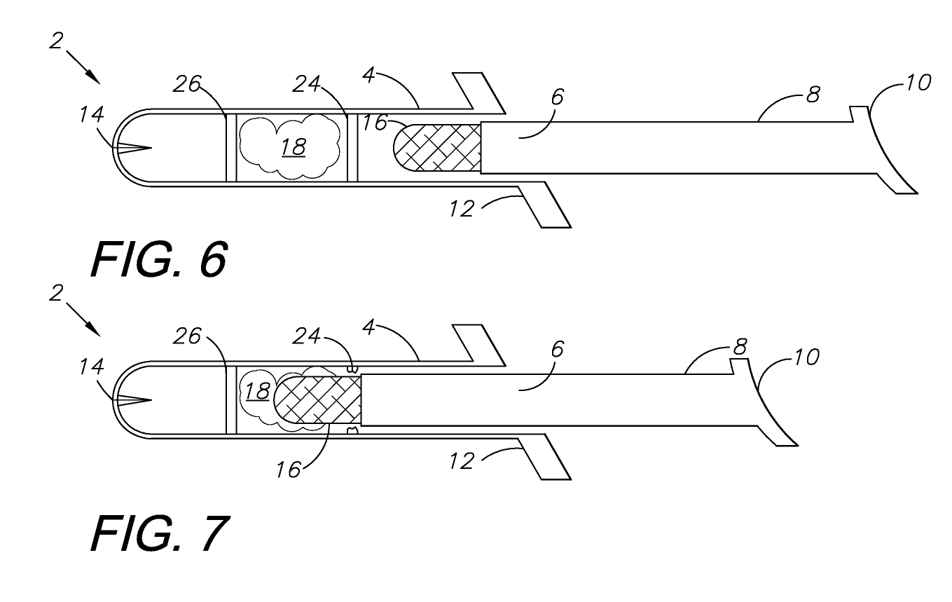

FIGS. 6-9 show a section of an embodiment of the present invention, and demonstrate a step-by-step method by which that embodiment may function. In FIG. 6, the applicator 2 is in its “starting position.” This will be the position the applicator 2 is in when it is removed from its packaging 3. A first seal 24 and a second seal 26 are located within the tube body. Between the first 24 and second 26 seals is a cream or liquid agent 18. These seals 24, 26 prevent the need for a cap, which may become clogged or be lost. The seals prevent the agent 18 from exiting the tube 4 until such a time that the seals are broken.

The agent is injected into the applicator 2 during manufacture, and may comprise any liquid or cream agent, such as a skin-soothing cream for use in treating diaper rash, cortisone for treating a skin itch, or any variety of skin soothing creams, balms, or ointments.

The seals are formed out of foil, lightweight plastic, or some other body capable of storing the agent 18 within the confines of the tube body 4, while allowing for easy penetration when force is applied to the seals 24, 26 by the applicator tip 16. The seals may optionally be perforated. In a preferred embodiment, the seals ensure that the cream or liquid agent 18 is kept sterile until the seal is perforated by the applicator tip 16.

FIG. 7 shows the single-use applicator 2 after the applicator tip 16 has pierced the first seal 24. The applicator tip 16 absorbs an amount of the agent 18 as it passes through. The applicator tip would typically be made of cotton or some other soft, semi-absorbent material.

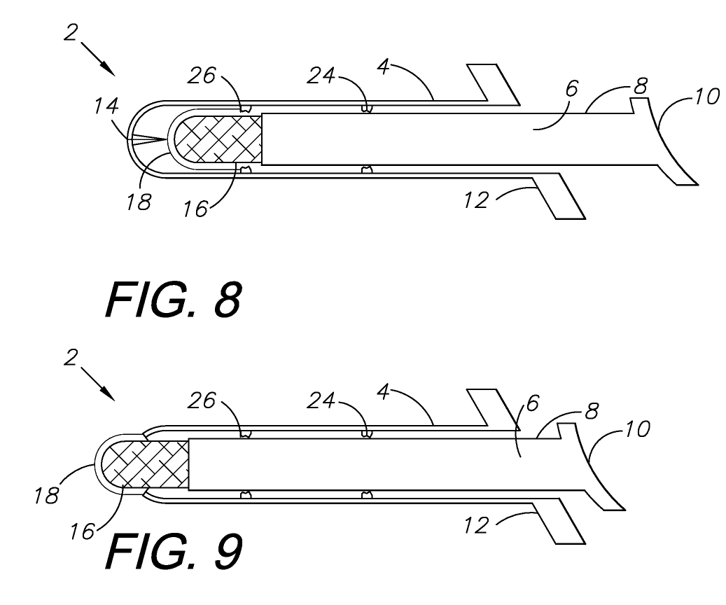

FIG. 8 shows the single-use applicator 2 after the applicator tip 16 has pierced the second seal 26. At this point during use, the applicator tip 16 is fully saturated with the cream or liquid agent 18 which was previously stored between the seals 24, 26 and the tube body 4.

FIG. 9 shows the single-use applicator in its “final position.” The applicator has pierced both seals 24, 26, and the applicator tips has emerged from the front opening enclosure 14 of the tube body 4. An amount of cream or liquid agent 18 remains coating and absorbed into the applicator tip 16. The tip may then be used to apply the agent 18 to a body. The tip 16 is soft so that it may be used to spread the agent 18 along a body with minimal discomfort. Once a desired amount of the agent 18 has been applied, the entire applicator 2 may be disposed. If additional applications are desired, the user may select another pre-wrapped applicator 7 from the storage box 5 and repeat the above steps.

It is to be understood that the invention can be embodied in various forms, and is not to be limited to the examples discussed above. The range of components and configurations which can be utilized in the practice of the present invention is virtually unlimited.

Having this described the invention, what is claimed as new and desired to be secured by Letters Patent is:

1. A dispenser and applicator apparatus comprising:
a hollow tube defining a volume, said tube including an inner circumference, and outer circumference, a proximal end and a distal end, said tube proximal end including a first opening, and said tube distal end include a second opening;
a plunger comprising a proximal end and a distal end, said applicator plunger distal end sized to the hollow tube inner circumference and adapted for being inserted into said tube first opening;
a first seal located within said hollow tube volume;
a fluid or viscous agent contained between said first seal, said plunger distal end, and said hollow tube inner circumference;
said first seal capable of breaking upon receiving pressure from said plunger; and
said plunger distal end adapted for receiving said liquid or viscous agent and spreading said liquid or viscous agent upon a desired surface.

2. The apparatus of claim 1, further comprising a second seal located within said hollow tube volume between said first seal and said applicator distal end, thereby containing said liquid or viscous agent between said first seal and said second seal.

3. The apparatus of claim 2, wherein said liquid or viscous agent remains sterile while contained between said first seal, said second seal, and said hollow tube inner circumference.

4. The apparatus of claim 3, further comprising;
said liquid or viscous agent comprising a substance capable of treating irritated or infected human skin; and
said desired surface comprising irritated or infected human skin.

5. The apparatus of claim 3, wherein:
said liquid or viscous agent is capable of treating diaper rash; and
said irritated or infected human skin suffers from diaper rash.

6. The apparatus of claim 2, wherein said first and second seals are comprised of perforated foil or a similar material.

7. The apparatus of claim 1, wherein said plunger distal end comprises a semi-absorbent material.

8. The apparatus of claim 1, further comprising;
an enclosure including an inner face and an outer face;
said enclosure capable of sealing said hollow tube front opening;
said enclosure being adapted to open when said plunger distal end applies pressure to said enclosure inner face; and
said enclosure being further adapted to allow said plunger distal end and liquid or viscous agent to exit said hollow tube when said enclosure has been opened.

9. The apparatus of claim 7, further comprising:
said enclosure including a plurality of flaps separated by incisions; and
wherein said enclosure forms a sterile, air-tight seal when closed.

10. The apparatus of claim 1, further comprising:
an ergonomic grip affixed to said hollow tube outer surface near said hollow tube distal end, said grip adapted to receive human fingers applying pressure laterally;
said plunger distal end adapted to receive a human thumb applying pressure laterally; and
wherein said plunger is capable of being forced through the defined volume of said hollow tube upon receiving lateral force upon said plunger distal end.

11. A dispenser and applicator apparatus comprising:
a hollow tube defining a volume, said tube including an inner circumference, and outer circumference, a proximal end and a distal end, said tube proximal end including a first opening, and said tube distal end include a second opening;
a plunger including a proximal end and a distal end, said plunger proximal end sized to the hollow tube inner circumference and adapted for being inserted into said tube first opening;
an ergonomic grip affixed to said hollow tube outer surface near said hollow tube distal end, said grip adapted to receive human fingers applying pressure laterally;
said plunger distal end adapted to receive a human thumb applying pressure laterally;
wherein said plunger is capable of being forced through the defined volume of said hollow tube upon receiving lateral force upon said plunger distal end;
said plunger distal end comprising a semi-absorbent material;
a first seal located within said hollow tube volume;
a second seal located within said hollow tube volume between said first seal and said hollow tube second opening;
a liquid or viscous agent comprising a substance capable of treating irritated or infected human skin contained between said first seal, said second seal, and said hollow tube inner circumference, wherein said agent remains sterile while contained between said first seal, said second seal, and said hollow tube inner circumference;
an enclosure including an inner face and an outer face;
said enclosure capable of sealing said hollow tube front opening;
said enclosure being adapted to open when said plunger distal end applies pressure to said enclosure inner face;
said first and second seals capable of breaking upon receiving pressure from said plunger;
said enclosure being adapted to allow said plunger distal end and liquid or viscous agent to exit said hollow tube when said enclosure has been opened; and
said plunger distal end adapted for receiving said liquid or viscous agent and spreading said liquid or viscous agent upon irritated or infected human skin.

12. The apparatus of claim 11, wherein:
said fluid or viscous agent is capable of treating diaper rash; and
said irritated or infected human skin suffers from diaper rash.

13. The apparatus of claim 11, wherein said first and second seals are comprised of perforated foil or a similar material.

14. The apparatus of claim 11, further comprising:
said enclosure including a plurality of flaps separated by incisions; and
wherein said enclosure forms a sterile, air-tight seal when closed.

15. A method of applying a liquid or viscous agent to a desired surface comprising the steps:
providing a storage body portion defining a volume, said storage body including a proximal end, a distal end, a first opening located near said storage body proximal end, a second opening located at said storage body distal end, an inner circumference, and an outer circumference;
providing an plunger including a proximal end and a distal end;
locating a first seal within said storage body volume sized to said storage body inner circumference;
inserting a liquid or viscous agent between said first seal, said second seal, and said storage body inner circumference;
inserting said plunger distal end into said storage body portion first opening, such that said plunger may move laterally within said storage body volume;
moving said plunger laterally through said storage body portion volume;
moving said applicator tip through said liquid or viscous agent, thereby displacing said agent within said plunger distal end;
moving said plunger distal end through said first seal, thereby breaking said first seal;
moving said plunger distal end through said storage body first opening; and
applying said liquid or viscous agent to a desired surface using said plunger distal end.

16. The method of claim 15, comprising the steps:
locating a second seal within said storage body volume between said first seal and said plunger distal end, thereby containing said liquid or viscous agent between said first seal and said second seal, said second seal sized to said storage body inner circumference;
moving said plunger through said second seal, thereby breaking said second seal.

17. The method of claim 16, wherein said first and second seals are comprised of perforated foil or a similar material.

18. The method of claim 15, wherein:
said fluid or viscous agent is comprised of a substance capable of treating irritated or infected human skin; and
said desired surface comprising irritated or infected human skin.

19. The method of claim 18, wherein:
said liquid or viscous agent is capable of treating diaper rash; and
said irritated or infected human skin suffers from diaper rash.

20. The method of claim 15, wherein said plunger distal end is comprised of a semi-absorbent material.

21. The method of claim 15, further comprising the steps;
providing an enclosure including an inner face and an outer face;
sealing said hollow tube front opening using said enclosure; and
moving said plunger distal end against said enclosure inner face, thereby opening said enclosure.

22. The method of claim 21, further comprising the steps:
providing said enclosure with a plurality of flaps separated by incisions; and
forming a sterile, air-tight seal with said enclosure.

23. The method of claim 15, further comprising:
providing an ergonomic grip affixed to said hollow tube outer surface near said hollow tube distal end, said grip adapted to receive human fingers applying pressure laterally;
providing pressure laterally against said plunger distal end and said ergonomic grip; and
forcing said plunger laterally through said storage body volume.
